(19) United States
(12) Patent Application Publication
(10) Pub. No.: US 2011/0071362 A1
(43) Pub. Date: Mar. 24, 2011

(54) RETRACTOR TOOL

(75) Inventor: Murray Reicher, Rancho Santa Fe, CA (US)

(73) Assignee: Health Beacons, Inc., Kirkland, WA (US)

(21) Appl. No.: 12/888,060

(22) Filed: Sep. 22, 2010

Related U.S. Application Data
(60) Provisional application No. 61/245,122, filed on Sep. 23, 2009.

(51) Int. Cl. A61B 1/32

(52) U.S. Cl. 600/217

(57) ABSTRACT

A retractor tool for retracting target tissue is provided for use with a medical probe. The retractor tool includes a guide piece that defines a probe channel and at least one retractor arm channel. Further, the retractor tool at least one retractor arm, each retractor arm passing through a corresponding retractor arm channel.

### PRIORITY CLAIM AND CROSS REFERENCE TO RELATED APPLICATIONS

This application claims the benefit of U.S. Provisional Application No. 61/245,122, filed Sep. 23, 2009.

### FIELD

A field of the invention is medical and surgical tools. Example applications of the invention include retaining tissue during a medical or surgical procedure.

### BACKGROUND

Surgical excision or exploration of tissue may be facilitated by various probe devices. The probe devices are generally cylindrical, and can be used for detection and differentiation of various tissues, or for detection of foreign objects that were previously inserted into the tissue. For example, a medical probe may be used to locate a radio frequency identification (RFID) chip or passive integrated transponder (PIT) tag embedded within target tissue. Alternatively, probes may be used in conjunction with measurement devices such as thermometers, Geiger counters, and other devices to collect information about the condition of the tissue at particular and relatively precise locations.

Once a probe device has identified a particular area of tissue that is of interest to the user, it is often necessary to retract the targeted tissue. However, a single user may find it difficult or awkward to use both a probe and a retractor at the same time. Alternatively, the process of locating target tissue with a probe, setting aside the probe to retrieve a separate retractor, and accurately retracting the target tissue identified by the probe is both difficult and time-consuming for users. Accordingly, there is a need for a retractor tool that can be easily and comfortably used in conjunction with a medical probe device.

### SUMMARY OF THE INVENTION

A retractor tool for retracting target tissue is provided for use with a medical probe. The retractor tool includes a guide piece that defines a probe channel and at least one retractor arm channel. Further, the retractor tool at least one retractor arm, each retractor arm passing through a corresponding retractor arm channel.

### BRIEF DESCRIPTION OF THE DRAWINGS

FIG. 1 is a perspective view of a retractor tool of some embodiments of the present invention;

FIG. 2 is an overhead plan view of a guide piece of the retractor tool of FIG. 1;

FIG. 3 is a perspective view of a guide piece with a probe channel locking mechanism;

FIG. 4 is an elevation of an embodiment of a guide piece, showing a probe channel axis and a parallel arm channel axis;

FIG. 5 is an elevation is an elevation of an embodiment of a guide piece, showing a probe channel axis and an intersecting arm channel axis; and

FIG. 6. is a perspective view of a retractor tool of another embodiment of the present invention.

### DETAILED DESCRIPTION OF THE PREFERRED EMBODIMENTS

Embodiments of the present invention provide a retractor tool for retracting targeted tissue. The retractor tool includes a guide piece that defines a probe channel and one or more retractor arm channels. Further, the retractor tool includes a medical probe that passes through the probe channel, and one or more retractor arms, each of the one or more retractor arms passing through a corresponding one of the one or more retractor arm channels.

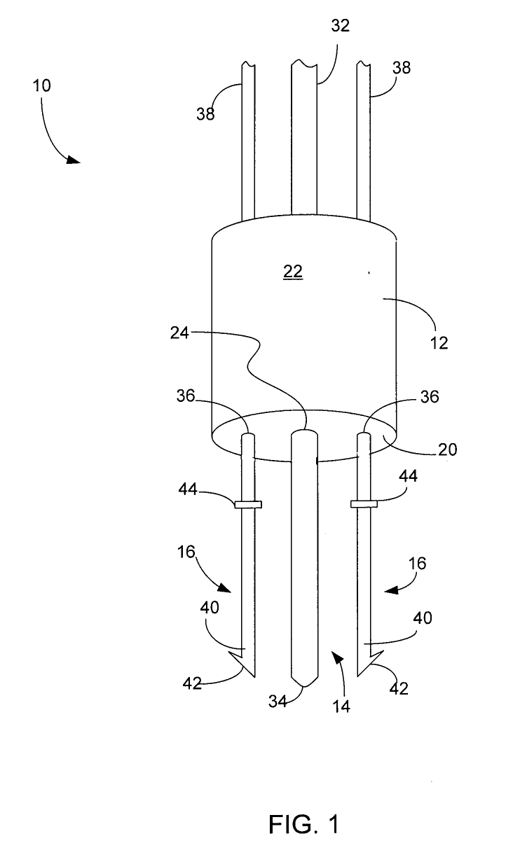

Referring now to FIG. 1, a retractor tool is generally designated 10. The retractor tool 10 includes a guide piece 12 used with a medical probe 14 and including one or more retractor arms 16. The guide piece 12 guides the positioning of the one or more retractor arms 16 relative to the medical probe 14.

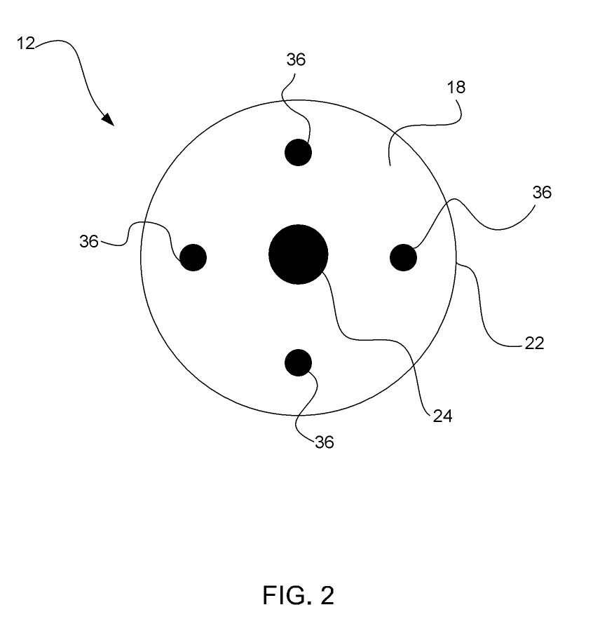

The guide piece 12 is preferably made from a single, solid piece of material, such as plastic, surgical stainless steel or aluminum, and may range in size from multiple millimeters to multiple centimeters. Alternatively, any material, such as a medical grade plastic, may be used to form the guide piece 12. The guide piece 12 includes a top face 18 as shown in FIG. 2, a bottom face 20, and a side wall 22 and is preferably generally cylindrical. Alternatively, the guide piece 12 may have any other three dimensional shape, or may be composed of multiple attached components that together provide for a channel for the probe 14 and one or more retractors arms 16.

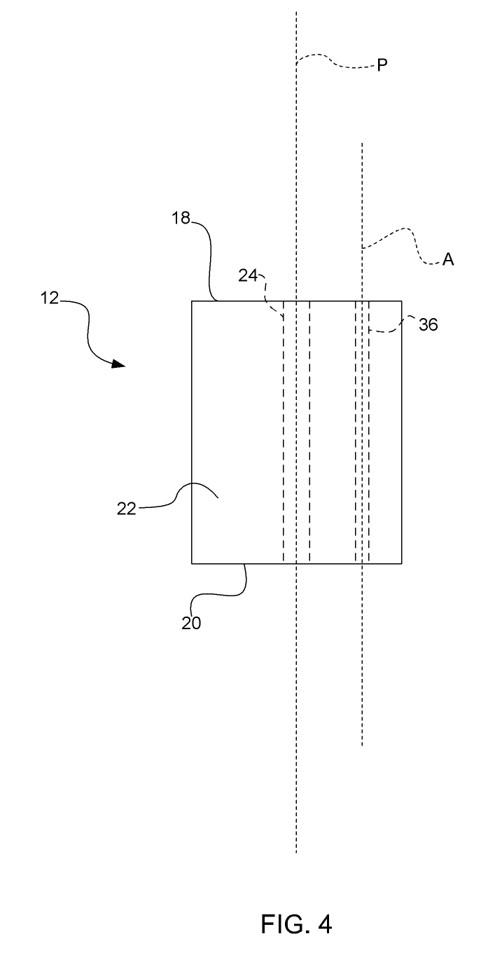

The guide piece 12 includes a probe channel 24 sized to allow the medical probe 14 to pass through the guide piece. The probe channel 24 preferably extends through the guide piece 12 in a direction that is generally perpendicular to the top and bottom faces 18, 20 of the guide piece, such that an axis P of the probe channel is substantially in parallel with an axis of the guide piece. Moreover, the probe channel 24 has a shape and size that are chosen to match the cross-sectional shape and size of the medical probe 14 with which the guide piece 12 is used. For example, when used with a medical probe having a generally circular cross-sectional shape, the probe channel 24 is generally cylindrical. Similarly, if the probe is not circular in cross-section, the guide channel 24 would not be circular, but would rather match the cross-sectional shape of the probe. Moreover, the probe channel 24 is sized so as to allow the guide piece 12 to move freely along the probe 14, sometimes including an accommodation for a sterile probe cover, and may or may not allow rotation about the axis P (FIG. 4) of the probe channel, while still maintaining a close fit. The probe channel 24 is preferably positioned at or near the center of the cross-section of the guide piece 12, but artisans will recognize that the probe channel could be positioned elsewhere on the guide without departing from the scope of the present invention.

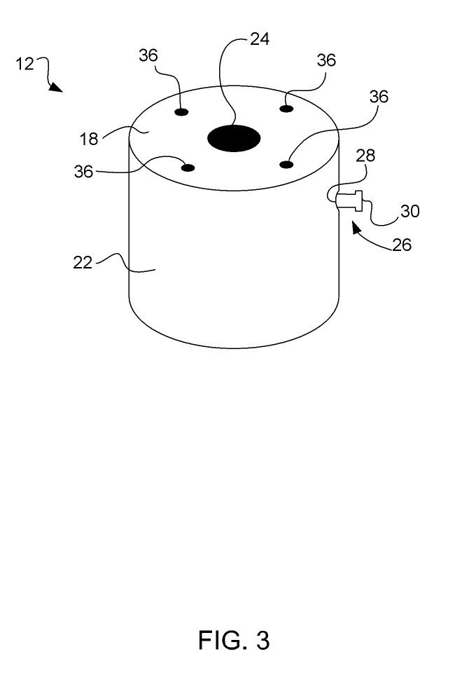

As shown in FIG. 3, some embodiments of the present invention include a guide piece 12 that has a locking mechanism 26 associated with the probe channel 24. The locking mechanism 26 releasably restricts movement of the guide piece 12 relative to the medical probe 14. For example, the guide piece 12 may include a clamping mechanism that restricts movement of the guide piece relative to the probe via friction force when the clamp is engaged. Additionally, such a clamping mechanism may also restrict rotation of the guide piece 12 about the probe channel axis P, relative to the probe 14. An example clamping mechanism 26 includes a threaded guide hole 28 positioned to receive a transverse screw 30 that passes through a portion of the guide piece 12 and into the probe channel 24, allowing the screw 30 to exert pressure on the probe 14 when a user tightens the screw.

Another possible locking mechanism 26 includes a stop positioned between the guide piece 12 and a proximal end 32 of the probe 14. The stop restricts movement of the guide piece 12 toward the proximal end 32 of the probe 14, while allowing for both movement of the guide piece toward a distal end 34 of the probe and rotation of the guide piece relative to the probe about the axis P.

Alternatively, an inner surface of the probe channel 24 could be manufactured to include threads. Then, at least a portion of the outer surface of the medical probe 14 is threaded to receive the guide piece 12. As another option, a threaded attachment may be mounted on a conventional probe 14 to receive the threaded probe channel 24. The mating threaded surfaces allow a user to adjust the linear position of the guide piece relative to the probe by rotating the guide piece.

Still other embodiments of the present invention include a guide piece 12 that is permanently affixed to the medical probe 14. The guide piece 12 may be affixed to the probe 14 using a variety of known attachment means, including welding, chemical adhesives, and the like. Additionally, the guide piece 12 may be integrally formed with the probe 14. Permanent affixation such as this permanently restricts movement of the guide piece 12 toward both the proximal and distal ends 32, 34 of the medical probe 14, and also prevents rotation of the guide piece relative to the probe.

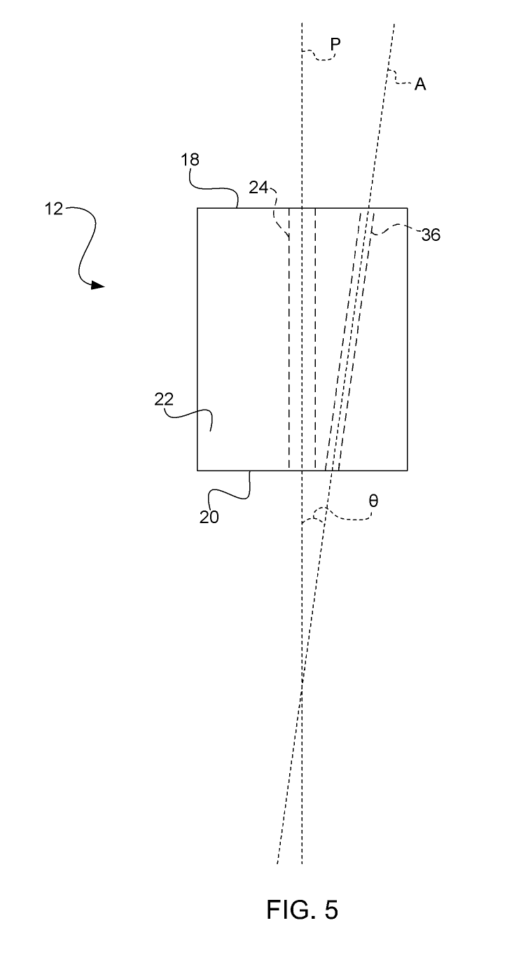

One or more arm channels 36 included in the guide piece 12 further enable passage of one or more retractor arms 16 through the guide piece. As examples, FIG. 1 shows a guide piece 12 including two arm channels 36, while FIGS. 2 and 3 each show a guide piece including four arm channels. Each arm channel 36 can be formed with an axis A that is generally parallel to the top probe channel 24, and thus is generally parallel with the probe axis P, as shown in FIG. 4. Alternatively, the axis A of each arm channel 36 may intersect with the probe channel axis P so that the axes A and P converge at a point external to the guide piece 12 forming an angle θ, either above the top face 18 or below the bottom face 20, as shown in FIG. 5. When the arm channel 36 has an angled axis A, the angle formed between the axis A of the arm channel and the axis P of the probe channel is preferably in a range of approximately 0° to approximately 30°. Thus, one or more of the retractor arms may be guided to converge or diverge relative to the probe's distal end.

The arm channels 36 are preferably spaced regularly in a circular pattern surrounding the probe channel 24. Alternatively, the pattern formed by the arm channels 36 may be the same shape as the top and bottom faces 18, 20 of the guide piece 12, or any other shape that is desirable for orienting the retractor arms 16. Further each of the arm channels 36 has a shape and size chosen to match the cross-sectional shape and size of the retractor arms 16, which preferably have a diameter in a range from less than 1 mm to several millimeters. That is, each of the arm channels 36 is sized such that a corresponding retractor arm 16 fits closely in the channel.

The medical probe 14 attached to the retractor tool 10 has proximal end 32 and distal end 34. The distal end 34 of the probe 14 includes a sensor (not shown) that monitors at least one input, as is known in the art. For example, the sensor may be an RFID reader, a Geiger counter, a metal detector, or any similar sensor useful for determining a location of target tissue for retraction, sampling, or the like. The proximal end 32 of the probe 14 is manipulated by a user such that the sensor is passed over a portion of tissue to determine a location of target tissue.

The retractor tool 10 further includes one or more retractor arms 16 that are used for restraining or retracting target tissue. The retractor arms 16 are preferably relatively long and thin, linear, wire-like parts having a generally cylindrical shape. Alternatively, the retractor arms 16 may be formed with other cross-sectional shapes, such as square, hexagonal, or relatively flat shapes. The arms 16 are preferably made from metals such as surgical stainless steel, but other rigid, resilient, biocompatible materials can be used in place of or in addition to metals to form the retractor arms. Each of the retractor arms 16 includes a proximal end 38 located near the user and a distal end 40 located in a position to restrain or retract target tissue. The distal end 38 preferably includes a retaining device 42 such as a barb as shown in FIG. 1. Alternatively, the retaining device may be a curved or angled portion, or the like that prevents target tissue from retuning to its original position until the user releases the tissue, or the entire retractor arm itself may be curved. Each retractor arm 16 can preferably be inserted into at least one arm channel 36 to allow for retracting of targeted tissue.

Once the retractor arm 16 is inserted into an arm channel 36, it may be necessary and/or beneficial to the user to lock the retractor arm in place, preventing movement of the arm toward one or both of the proximal and distal ends 32, 34 of the medical probe 14. Accordingly, some embodiments of the retractor tool 10 may optionally include a locking mechanism having locked and unlocked positions. The locking mechanism releasably restricts movement toward both the proximal and distal ends of the medical probe when the mechanism is in the locked position, and does not restrict movement of the arm relative to the probe when the mechanism is in the unlocked position.

Alternatively, as shown in FIG. 1, some embodiments of the retractor arm 16 include a stop 44 disposed between the proximal and distal ends 38, 40 of the retractor arm. The stop 44 extends radially from a surface of the retractor arm 16 and is used to control or limit the movement of the retractor arm 16 relative to the guide piece 12. Specifically, the stop 44 helps to prevent the retractor arm 16 from travelling past a predetermined point toward the proximal end 32 of the probe 14 while not restricting movement toward the distal end 34 of the probe. The stop 44 may be a metal or plastic protrusion that has a diameter larger than that of the arm channel 36. The stop 44 is attached to the arm 16 using an adhesive, via a welding process, or by being formed integrally with the arm.

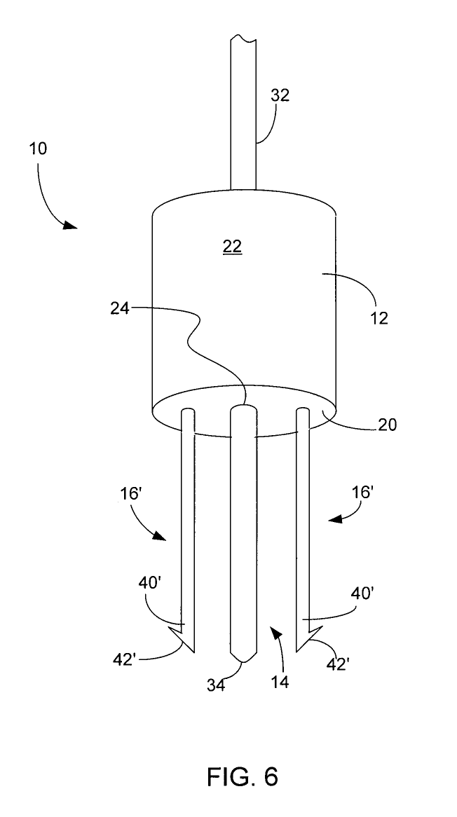

As shown in FIG. 6, another embodiment of the retractor tool 10 may include retractor arms 16′ that are permanently affixed to the guide piece 12. That is, each arm 16′ may be connected to the guide piece 12 by a welded joint, a chemical adhesive, by being formed integrally, or the like. Each of the retractor arms 16′ also includes a retaining device 42′ such as a barbed tip at a distal end 40′ of the arm. When the retractor arms 16′ are permanently affixed to the guide piece 12, the arms are permanently restrained from moving towards both the distal end 32 and the proximal end 34 of the probe 14.

In use, the medical probe is inserted through the probe channel and one or more retractor arms are inserted into corresponding arm channels as desired by the user. The user then operates the probe according to known techniques to locate target tissue. Once target tissue has been located, the tissue is gripped for retraction.

Retracting tissue can be accomplished in various ways. For example, the entire retractor tool including the probe, guide piece, and all retractor arms may be moved toward the target tissue as a single unit. Alternatively, the guide piece and retractor arms may be moved toward the distal end of the probe while the probe is held in a stationary position, or one or more individual retractor arms may be moved toward the distal end of the probe while the probe and the guide piece are held stationary. Each of these methods causes one or more retractor arms to become embedded in the target tissue, allowing the user to manipulate the tissue as required through lateral movement and/or rotation of the retractor tool, guide piece, and/or individual retractor arms. The retaining devices located at the distal end of each retractor arm helps to prevent the target tissue from returning to its initial position until the user no longer requires that the tissue be retracted. In these ways, the user can control the amount and location of target tissue that is retracted by the arms.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

1. A retractor tool for retracting target tissue, the retractor tool comprising:
a guide piece defining a probe channel and at least one retractor arm channel; and
at least one retractor arm, each retractor arm passing through a corresponding retractor arm channel.

2. The retractor tool of claim 1, wherein the probe channel is threaded.

3. The retractor tool of claim 1, wherein the probe channel has a non-circular cross-section.

4. The retractor tool of claim 1, wherein the at least one retractor arm channel has an axis that is not parallel to the probe channel.

5. The retractor tool of claim 1, wherein the at least one retractor arm channel has an axis that is substantially parallel with the axis of the probe channel.

6. The retractor tool of claim 1, wherein the at least one retractor arm includes a barbed tip.

7. The retractor tool of claim 1, wherein the at least one retractor arm has one of a curved shape and an angulated shape.

8. The retractor tool of claim 1, wherein the at least one retractor arm is non-circular in cross-section.

9. The retractor tool of claim 1, wherein the at least one retractor arm and the guide piece are made from the same material.

10. The retractor tool of claim 1, further comprising:
a threaded channel extending transversely across a portion of the guide piece between the side wall and the probe channel; and
a threaded screw sized to mate with the threads of the threaded channel, the screw having a length that exceeds a length of the threaded channel.

11. The retractor tool of claim 1, further comprising a medical probe extending through the probe channel and a locking mechanism on the guide piece configured to engage the medical probe for restricting movement of the guide piece relative to a medical probe.

12. The retractor tool of claim 1, in which the guide piece is comprised of multiple components that together define the probe channel.

13. The retractor tool of claim 1, wherein the at least one retractor arm includes a stop radially extending from a surface of the at least one retractor arm and configured to limit movement of the at least one retractor arm relative to the guide piece.

14. A retractor tool for retracting target tissue, the retractor tool comprising:
a guide piece defining a probe channel; and
at least one retractor arm, each retractor arm being fixed to the guide piece.
